(19) United States
(12) Patent Application Publication
(10) Pub. No.: US 2013/0131555 A1
(43) Pub. Date: May 23, 2013

(54) GAIT ANALYSIS USING ANGULAR RATE REVERSAL

(71) Applicants: William R. Hook, Shawnigan Lake (CA); Christopher R. Harris, Vancouver (CA); Sunny V. Mahajan, Victoria (CA)

(72) Inventors: William R. Hook, Shawnigan Lake (CA); Christopher R. Harris, Vancouver (CA); Sunny V. Mahajan, Victoria (CA)

(21) Appl. No.: 13/675,951

(22) Filed: Nov. 13, 2012

Related U.S. Application Data
(60) Provisional application No. 61/561,152, filed on Nov. 17, 2011.

(51) Int. Cl. A61B 5/11

(52) U.S. Cl. CPC A61B 5/112
USPC 600/595

(57) ABSTRACT

The gait of a subject can be assessed based on angular rate reversals in one or more limbs. Angular rotation sensors are secured to the limb and the associated data is processed to determine angular rate reversals. Alternatively, image capture systems can be used to identify rate reversals. Based on two or more rate reversals, subject gait and gait characteristics can be evaluated, even for subjects having shuffling gaits.

### CROSS REFERENCE TO RELATED APPLICATION

This application claims the benefit of U.S. Provisional Application 61/561,152, filed Nov. 17, 2011, which is incorporated herein by reference.

### FIELD

The disclosure pertains to methods and apparatus for gait analysis.

### BACKGROUND AND SUMMARY

While the determination of gait parameter for non-disabled walkers can be straightforward, gait analysis for those individuals for whom gait analysis could be most helpful is generally not available with conventional approaches. Disabled individuals may have irregular or shuffling gaits that lack the typical features used for conventional analysis. Methods and apparatus are disclosed herein that can provide suitable gait analysis for all individuals, including severely disabled individuals.

Methods of gait analysis comprise obtaining an angular rate of a subject limb as a function of time during the gait of the subject. At least two angular rate reversals during the gait are identified based on the obtained angular rate, and at least one gait characteristic is estimated based on the two such angular rate reversals. In some examples, the at least two angular rate reversals are identified based on a rate of change of the angular rate. In particular examples, the rate of change is at least 80% of a maximum rate of change or a maximum rate. In typical examples, the gait characteristic is associated with initiation of a gait cycle.

Apparatus comprise at least one sensor configured to provide an angular rate associated with a limb or portion of a limb such as a shank at a plurality of times during at least one gait cycle. A processor is configured to receive the angular rate at the plurality of times and produce angular rate data. Based on a rate reversal in the angular rate data, a gait cycle initiation time is estimated. In some examples, the at least one sensor is a gyroscopic sensor configured to be secured to the shank. In other examples, the sensor is an image capture sensor configured to produce the angular rate at the plurality of times based on a series of images of at least the shank. In further examples, the processor is configured to estimate at least one gait characteristic based on the gait cycle initiation times. In some examples, the at least one gait characteristic is a stride length, stride duration, or a limb range of motion or a variation thereof.

Methods comprise obtaining angular rate at a plurality of times for a limb, and based on the obtained angular rate, identifying at least two angular rate reversals. Based on the identified angular rate reversals, an assessment of a subject is provided. In some examples, the assessment of the subject is based on a variation in gait cycle initiation times associated with corresponding angular rate reversals. In some examples, means or other moments or statistical properties of distribution of such parameters are obtained in order to assess performance.

In some disclosed examples, methods of assessing cyclical motion comprise obtaining an angular rate associated with the motion for at least one cycle, identifying at least two angular rate reversals about a threshold value, and characterizing the motion based on the identified angular rate reversals about the threshold value. In typical examples, the identified angular rate reversal about a threshold value is associated with a forward gait, and an initiation time of a forward swing is identified based on the angular rate reversal about the threshold. In other examples, termination of the forward swing is identified based on an angular rate that is derived from a maximum angular rate in the same gait cycle and subsequent to the angular rate reversal about the threshold. In still further examples, a toe lift is identified based on an angular acceleration that is derived from a maximum angular acceleration in the same gait cycle and prior to the angular rate reversal about the threshold.

In other examples, methods of assessing fall risk include obtaining an angular rate for a subject during a plurality of gait cycles and identifying threshold crossings of the obtained angular rate. A gait characteristic for the plurality of gait cycles is estimated for each of the plurality of gait cycles and the estimated gait characteristic for the plurality of gait cycles is compared to a predetermined gait characteristic value. In typical examples, the gait characteristic is stride length or stride time. In some specific examples, the variation of the gait characteristic such as a standard deviation is obtained.

In other examples, an apparatus include at least one sensor configured to provide at least one of an angular acceleration or an angular speed associated with a shank at a plurality of times during at least one gait cycle. A processor is configured to receive at least one of the angular speed or the angular acceleration at the plurality of times, and based on the application of an angular acceleration threshold, estimate a stride event point time. In some embodiments, the at least one sensor is a gyroscopic sensor configured to be secured to the shank, and the processor is configured to produce angular acceleration data by differentiating the angular rate data from the gyroscopic sensor. In further examples, the sensor is an image capture sensor configured to produce a series of images of at least the shank, and provide the angular acceleration or the angular speed based on the images. In additional embodiments, the processor is further configured to estimate at least one gait characteristic based on the detection of stride event point times, and the at least one gait characteristic is a stride length, stride duration, or a limb range of motion. In still further embodiments, the at least one gait characteristic is a variation in at least one gait property.

The foregoing and other features of the invention will become more apparent from the following detailed description, which proceeds with reference to the accompanying figures.

### BRIEF DESCRIPTION OF THE DRAWINGS

FIG. 1 is a graph illustrating a gait cycle illustrating toe off and heel strike portions.

FIG. 2 is a gait cycle graph illustrating identification of gait events in association with angular rate reversals.

FIG. 3 is a block diagram of a representative method of gait analysis.

FIG. 4 illustrates angular rate reversals during a walk by a representative subject.

FIG. 5 is graph of the gait data of FIG. 2 with decision points re-labeled to indicate variations in threshold values.

FIG. 6A is a graph illustrating the setting of alternative threshold values for the determination of angular rate reversals with respect to the alternative thresholds.

FIG. 6B is a graph illustrating the use of angular acceleration for determining the steepness of the angular rate slope data, or for setting an alternative angular rate threshold.

FIG. 7 is a schematic diagram of a representative gait detection system.

FIGS. 8A-8C illustrate processing of gait data.

FIG. 9 is a block diagram of a representative hardware system for acquisition of gait data.

FIG. 10 is a block diagram of a representative computing environment for implementation of the disclosed technology.

FIG. 11 illustrates representative gait data for 3 persons with Parkinson's disease in exercise classes.

FIG. 12 illustrates representative gait data for 2 persons with various types of shoes.

FIG. 13 is a gait cycle graph obtained from a dog.

FIG. 14 is a picture illustrating sensor placement on a dog used in obtaining the data of FIG. 13.

FIG. 15 is a table listing representative applications of the disclosed methods and apparatus.

### DETAILED DESCRIPTION

As used in this application and in the claims, the singular forms “a,” “an,” and “the” include the plural forms unless the context clearly dictates otherwise. Similarly, the word “or” is intended to include “and” unless the context clearly indicates otherwise. The term “comprising” means “including;” hence, “comprising A or B” means including A or B, as well as A and B together. Additionally, the term “includes” means “comprises.”

The disclosed methods, apparatus, and systems should not be construed as limiting in any way. Instead, the present disclosure is directed toward all novel and nonobvious features and aspects of the various disclosed embodiments, alone and in various combinations and subcombinations with one another. The disclosed methods, apparatus, and systems are not limited to any specific aspect or feature or combination thereof, nor do the disclosed embodiments require that any one or more specific advantages be present or problems be solved.

Theories of operation, scientific principles or other theoretical descriptions presented herein in reference to the apparatus or methods of this disclosure have been provided for the purposes of better understanding and are not intended to be limiting in scope. The apparatus and methods in the appended claims are not limited to those apparatus and methods that function in the manner described by such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

In some examples, the disclosure pertains to gait analysis systems and methods that include one or more devices for measuring the angular rate of at least two human limb segments, and a processing component, wherein one such limb segment is the shank. Further, such devices for determining the angular rate of each human limb segment can be based on body-mounted sensors or remote sensors or sensing systems such as optical motion capture systems employing either passive or active markers attached to the limb segments, and where the processing component is synchronized to the gait cycle by detecting the angular rate reversals of the shank.

As used herein, gait refers to a periodic or cyclical motion of a limb or other body part of a human or other subject. In the disclosed examples, particular emphasis is provided on characterization of the walking gait of human subjects in view its practical and economic significance, but the disclosed methods and apparatus are generally applicable to the motions of other human body parts, or the motions of animals.

The following terms are used in description of gait, particularly walking or running. A stride event point time is defined as a unique time associated with a particular event within a gait cycle, a limb refers to an arm, a leg, a head, or other extremity, and a swing phase of a gait cycle is defined at that portion of a gait cycle wherein the toe has lifted off the ground and the subject has begun the act of swinging forward. For normal walking, the swing phase begins with a short backward swing of the shank (characterized by a negative angular rate), and then starts a forward swing at the point where the angular rate changes from negative to positive (referred to herein as “initiation of forward swing”); the swing phase terminates when the shank angular rate changes from positive to negative (referred to herein as “termination of forward swing”); for a normal subject, physical heel strike occurs just after termination of forward swing; for a subject with a shuffling gait, and whose feet rarely or never leave the ground, the swing phase begins with the initiation of forward swing, and ends with the termination of forward swing. The swing phase may be characterized as a time duration (swing time) or normalized as a percent of the full gait cycle. A stance phase of a gait cycle refers to that portion of a normal gait cycle in which the foot is on the ground. In normal walking, the stance phase begins with the “termination of forward swing” and ends when the toe lifts off the ground. The stance time is usually calculated as the stride time minus the swing time expressed as a time interval, or normalized as the stride time minus the swing time divided by the stride time, expressed as a percent of the gait cycle. For a person with a shuffling gait, the stance phase is a time interval in which the foot is stationary on the ground. A gait cycle starts and ends with a particular stride event point time defined herein as a gait cycle initiation time.

In one example, systems and methods are based on at least two body-mounted sensors and a processing component, wherein each sensor includes at least an angular rate sensing device. The first sensor is attached to the shank of one leg. The second sensor may be attached to another body part, such as the thigh of the same leg; additional sensors may be attached to other body parts. The processing component is synchronized to the walking cycle by detecting the angular rate reversals indicated by the first sensor. The processing component is configured to calculate gait stride time, stride length, limb segment and joint angle parameters for one or more for each walking stride, as well as resultant statistics for a sequence of strides, including fall prediction parameters. This system is designed to measure small changes in walking parameters as a result of treatment, stress or time, or in the evaluation of walking aids such as a cane, walker, or passive and active prosthetics, or in the evaluation of footwear, or in the evaluation of the effect of uneven and irregular walking surfaces. Such systems can be used in clinical or research applications, where the subject walks in a generally straight line with a pause at the start and at the end, or in the home or outside with spontaneous walking. When used with wireless or internal data storage sensors, such a system can measure a statistically significant number of strides in sequence to provide superior statistical results for all walking parameters when compared to prior art fixed location gait analysis methods such as those using cameras or pressure plates. The disclosed gait analysis systems can be easy to use, portable, and less costly than commercially available systems. Moreover, the disclosed systems and methods can provide reliable assessments of a wide range of walking subjects, from those who are able-bodied to those with disabilities such as subjects with Parkinson's disease or Cerebral Palsy who walk with a shuffling gait, subjects for whom prior art gyro-based systems tend to be unreliable.

In other examples, optical body motion capture systems are used to establish limb angular rates. For example, angular rate can be obtained by differentiating the record of limb segment angle derived from two or more passive or active sensors attached to such limb segment, wherein the limb segment is one shank.

Body mounted sensors are typically inertial measurement units (IMUs), wherein the angular rate sensing device is a MEMS gyro. Usually an IMU is also provided with a gravity sensing device such as MEMS accelerometer, and a North sensing device such as a MEMS magnetometer. Either wired or wireless sensors can be used, and sensors can include data storage.

For continuous walking applications, an IMU gravity sensing device (a MEMS accelerometer) is not reliable for the determination of individual stride statistics due to the large and continuous external forces of acceleration provided by the leg movement, and thus cannot be used to provide reliable and accurate gait timing data for individual strides. Nor can it be used to periodically reset the gyro integration function in order to overcome the well-known “angle random walk” problem. Thus, the prior art IMU accelerometer-gyro fusion algorithm data processing methods favored by many researchers and commercial IMU organizations cannot be used to obtain accurate gait timing, limb and joint angle, balance and fall prediction data in a continuous walking application, even if the subject walks in a perfectly straight line.

An important practical portion of the processing described in this disclosure is the ability to determine when walking is not occurring (that is, when the subject is standing in a stationary position). For that function, the accelerometer can be and is used effectively in the processing of the data in a typical clinical measurement. Finally, in the normal clinical or home setting, the North sensing device (a MEMS magnetometer) is highly inaccurate due to the influence of nearby metallic objects of all sorts, and need not be used at all in the processing described in this disclosure.

In response to these problems, gait analysis systems based on the gyro output data during continuous walking have been developed. These systems have a number of significant features, including:
1. Use of output data from one of the three axes available from a 3-axis gyro, wherein that axis is aligned as best as possible to be vertical to the plane of swing of the leg, commonly called the sagittal plane.
2. Identifying the instants (in time) when one gait cycle starts and ends using only the gyro data. This allows for the calculation of “stride time” and facilitates a host of other calculations. This is often the most important direct measurement in a gait analysis application. In virtually all such systems, including the present disclosure, this event is associated with the physical “heel strike” of normal walking, occurring near the end of the “swing phase” of the lower leg limb (shank) when the foot first hits the ground after swinging forward.
3. Identifying the instant (in time) when the foot lifts off the ground and begins the swing phase.
4. Measuring limb and joint angles at every point in the gait cycle for determining the range-of-motion of limb segments and joints.
5. Calculating the stride length.

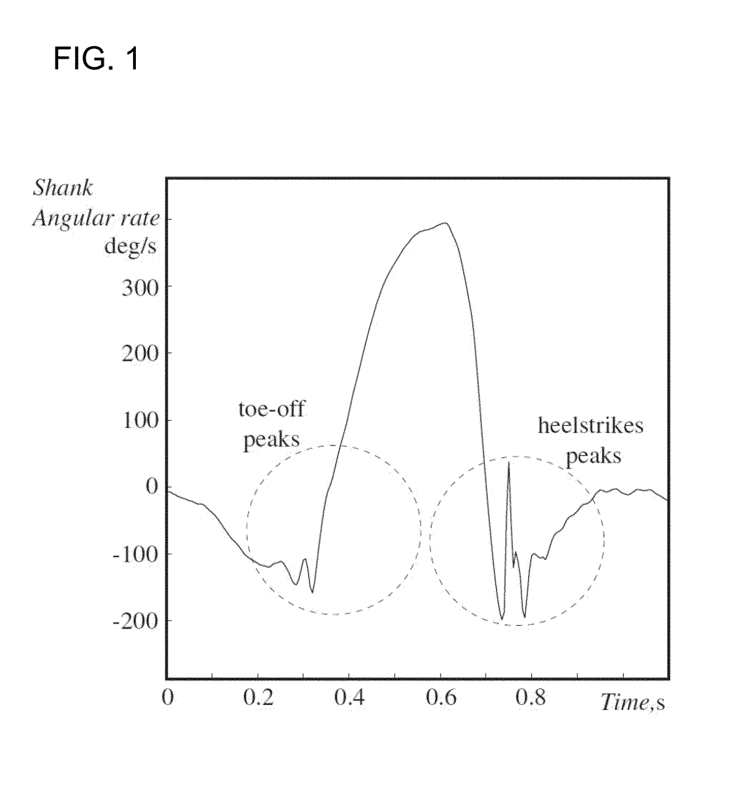

In one gyro-based prior art gait analysis system (Aminian, U.S. Patent Application Publication 20050010139, which is incorporated herein by reference), these five features were implemented as follows:
1. Mount IMU on the front of the shank (ankle) with active gyro axis vertical to the direction of walking.
2. Start of gait cycle: Use the large negative peak of the gyro output just following the swing phase of the shank (see FIG. 1).
3. Foot lift off ground: Use the large negative peak of the gyro just preceding the swing phase of the shank (see FIG. 1). For a non-disabled person, this typically occurs at about the 60% point in the gait cycle.
4. Limb and joint angles: Integrate the gyro output over each gait cycle in order to determine the difference between the minimum and the maximum angle, and declare that to be the range of motion (ROM), where the gyro integrator is reset to zero at the start of each gait cycle.
5. Stride length: Use the methods and corrected equations from Aminian, et al., “Spatia-temporal parameters of gait measured by an ambulatory system using miniature gyroscopes,” Journal of Biomechanics 35:689-699 (2002), which is incorporated herein by reference.

Unfortunately, these methods exhibit several undesirable properties:
A. The negative peak method for determining the start of the gait cycle, as described in item #2 above, becomes unusable due to excessive errors in determining the start time of each gait cycle for the important class of disabilities where the tested person has a shuffling gait. Note that for a shuffling gait, the feet rarely leave the ground, and so there is no such thing as a physical “heel strike”, and thus no mechanism for creating the desired negative peak.
B. The negative peak method for determining the start of the gait cycle, as described in item #2 above, becomes less accurate as the walking disability of the person being tested increases until it becomes unusable as stated above.
C. In order to achieve acceptable accuracy for the measurement of item #2 above, it is necessary to employ a 200 Hz data sampling rate. But normal body motion monitoring requires no more than a 40 Hz data sampling rate. This 200 Hz data rate requires a higher frequency, shorter range radio for wireless applications (as compared to an IMU with a 40 Hz data rate), and creates five times as much data as is necessary.

Some examples described herein overcome one or more or all of these limitations.

Conventional image capture based systems require pressure plates for determining the first 2 or 3 heel strikes, and then use image correlation methods for subsequent heel strikes, wherein images from the first two heel strikes are correlated with those from subsequent heel strikes. Thus these systems fail in the same way that conventional pressure plate systems fail (such as the GAITRITE system). That is, the pressure plate methods do not work reliably for persons with a shuffling gait, wherein the foot rarely leaves the ground, since it is dependent on the physical heel strike. This characteristic excludes significant numbers of severely disabled persons. The apparatus and methods disclosed herein can reduce or eliminate this limitation.

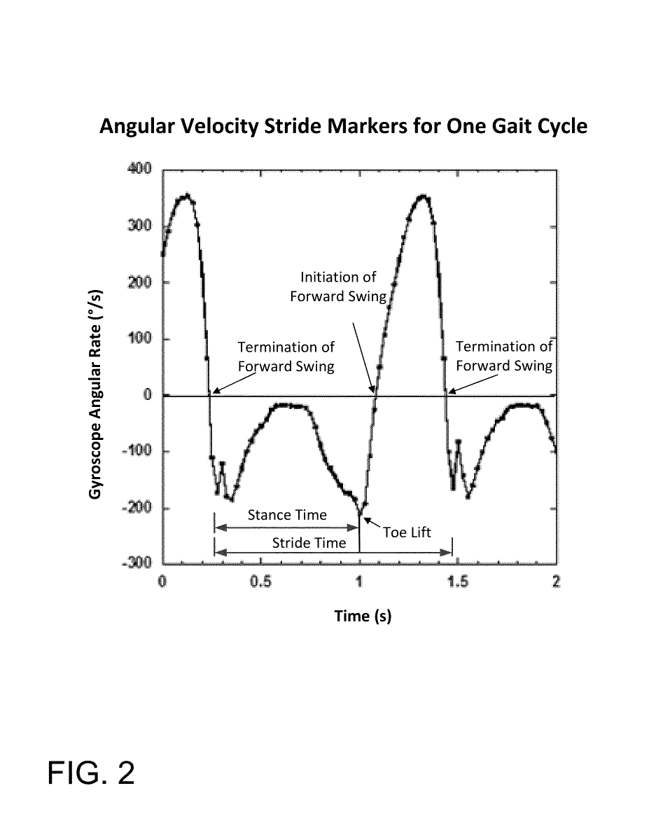

In some examples, the methods and apparatus disclosed herein provide more accurate and reliable statistical gait analysis data for clinical and research walking-disability applications by providing improved methods for determining the start of each or one or more gait cycles. FIG. 2 illustrates event timing in a gait cycle based on shank angular rates obtained with body mounted sensors. In some embodiments, the start of the gait cycle for each stride from angular rate data is based on the detection of the negative-going angular rate reversal point during each gait cycle, as shown in FIG. 2 (labeled “Termination of Forward Swing”). The angular rate can be determined based on body mounted sensors in a body-mount embodiment or on motion analysis with a camera system that does not require body mounting. The negative-going rate reversal point is a highly accurate measurement of the instant the shank ends the forward direction and begins a backward angular motion (termination of forward swing). This negative-going rate reversal is associated with a heel strike, is very close in time to the time of physical heel strike, and is defined as the start and end point of the stride time for each stride in our processing method. Timing within a gait cycle is conveniently described with respect to a normalized stride times which are expressed as a percentage of a total gait cycle time. Thus, the timing of events which occur between the negative rate reversal points is described in terms of percentage of the total gate cycle time.

A positive-going rate reversal point is associated with initiation of forward swing during the swing phase, and is labeled “Initiation of Forward Swing” in FIG. 2. This usually occurs at between 68-72% of the gait cycle, and can be useful in certain situations with severely disabled persons. Timing data can be derived from angular rate reversals at the two points in the gait cycle wherein the rate of change of the angular rate output is at or near a maximum, such as 60%, 80%, 90%, 95%, or 97% or more of a gait maximum which and can be 2,400°/s² or more during walking tests.

For example systems based on a 40 Hz sample rate, linear interpolation can be used between the 25 ms sample intervals to assign event times. If one sample is above zero rate (positive) and the next sample is below zero rate (negative), a basic system accuracy of about 5 ms is obtained. Physical toe lift (i.e., start of swing phase) typically occurs at a time that is approximately 10% of the gait cycle time earlier than the positive rate reversal time, so the leg angle moves backwards during the first portion of leg swing. The method of Salarian et al., “Gait Assessment in Parkinson's Disease: Toward an Ambulatory System for Long-Term Monitoring,” IEEE Trans. Biomed. Eng. 51:1434-1443 (2004), which is incorporated herein by reference, can be used to estimate the instant of toe lift (i.e., the start of swing phase). This method depends on finding the exact location of a local negative peak located just prior to forward swing. Unfortunately, this toe lift peak is sometimes wide, uneven and unpredictable. Although the toe lift peak is less accurately defined than the positive-going rate reversal point, this peak provides a reasonable estimate of physical toe-lift, and can be used to calculate stance time. For severely disabled walkers having a shuffling gait, and for whom toe lift peak is very poorly defined and has little physical meaning, the positive going rate reversal (which occurs at the start of forward swing, as shown on FIG. 2) can be used to define the termination of the “effective stance” phase and is labeled “Initiation of Forward Swing” on FIG. 2.

For body mounted sensor systems, the angular rate data can be derived directly from a gyro attached to a limb, typically the shank. For optical motion capture systems, angle data as a function of time is obtained from motions of 2 or 3 active or passive reflectors attached to the limb in question, again typically the shank, and then differentiated to obtain the limb angular rate data.

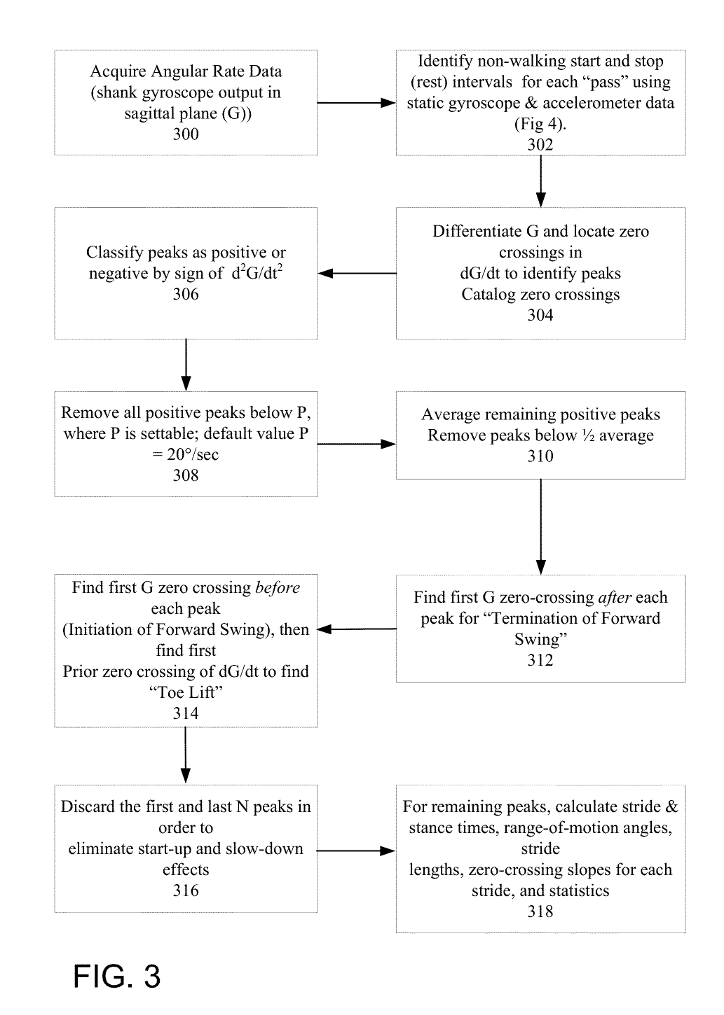

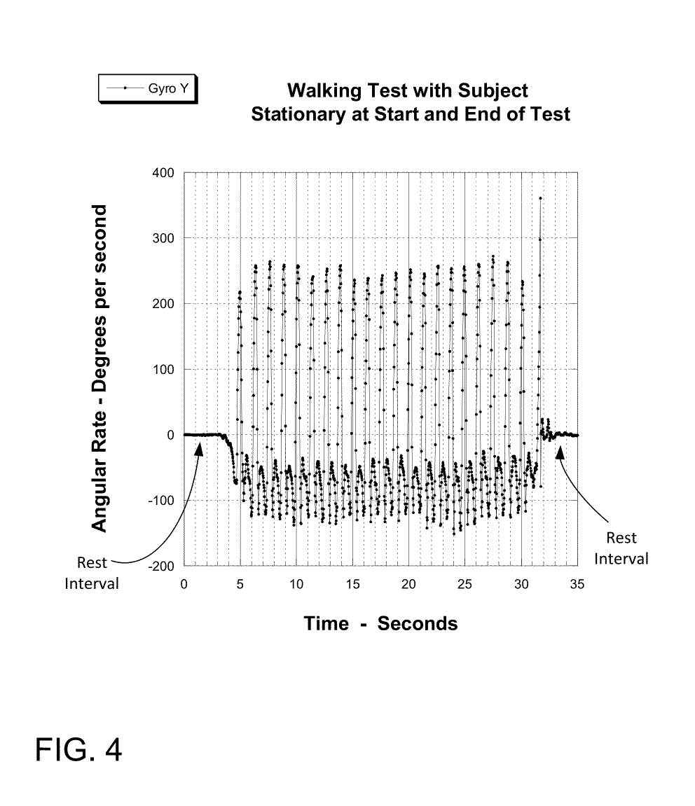

FIG. 3 illustrates a representative temporal gait event detection process. At 300, angular rate data such as shown in FIG. 4 are obtained. At 302, rest intervals at the start and the end of a subject evaluation are identified as shown in FIG. 4 based on the measured angular rate data. Characteristics of a plurality of gait cycles can be obtained using both the negative and the positive-going zero crossings from the angular rate data can be used. For example, the negative-going crossing, labeled “termination of forward swing” in FIG. 2 can be used to determine the start of each gait cycle, and thus can be used to determine stride time for each stride and thus stride time variation. The positive-going crossing, labeled “initiation of forward swing”, allows a reliable determination of “Toe Lift” as the first negative peak prior to initiation of forward swing for persons with most levels of walking disability. For persons with a shuffling gait who have no physical toe lift, the positive-going crossing labeled “initiation of forward swing” can be used as an approximation of toe lift. The toe lift point is used to divide each stride into a “stance” and “swing portion. The angle change during each portion of each stride is obtained by integrating the gyroscope output G based on shank mounted gyroscope output in a sagittal plane. This can be done for both the shank and the thigh, and produces the four required inputs for the Aminian stride length equations. At 304, G is differentiated to locate one or more (or all) zero crossings in dG/dt to identify peaks, and the results are cataloged or stored. At 306, peaks are classified as positive or negative based on the sign of the second derivative of G. At 308, some or all positive peaks are removed based on a settable value P, wherein a default value of P is 20 degrees/sec. At 310, the remaining positive peaks are averaged and all peaks below ½ average of remaining peaks are removed or not depending on a user selection. At 312, first zero-crossings in G after each peak are found for “Termination of Forward Swing” using linear interpolation of sample values just above and just below G=0 or as otherwise determined. At 314, first G zero crossings before each peak (Initiation of Forward Swing), and then first zero crossings of dG/dt before respective G zero crossings are found, using linear interpolation or other processes to obtain “Toe Lift.” At 316, first and last N peaks can be discarded to eliminate start-up and slow-down effects. At 318, for the remaining peaks, stride and stance times, range-of-motion angles, stride lengths, zero-crossing slopes for each stride, and stride statistics are calculated.

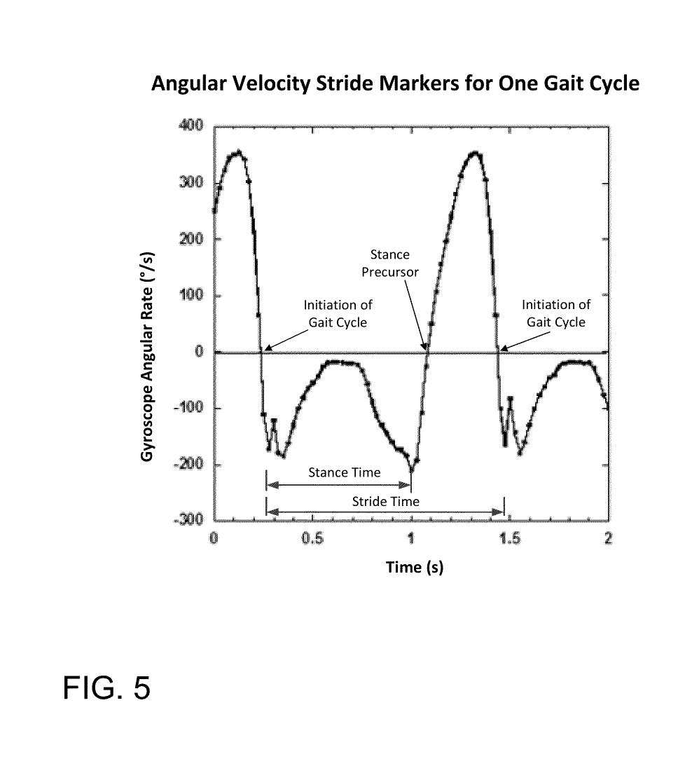

Although gait event detection can be conveniently determined based on a zero rate, other event thresholds can be used. FIG. 5 is a graph of the same information as FIG. 2, but wherein the various decision points are re-labeled to show the more general nature of those points. In this representation, the negative-going zero crossings are labeled with the properly more general terms “Initiation of Gait Cycle,” defining the length of each stride. The positive-going zero point is labeled as the “Stance Precursor” in recognition of the fact that, for the stance period, this point is identified prior to the identification of the end of the stance period as being the first negative peak just prior to the Stance Precursor point.

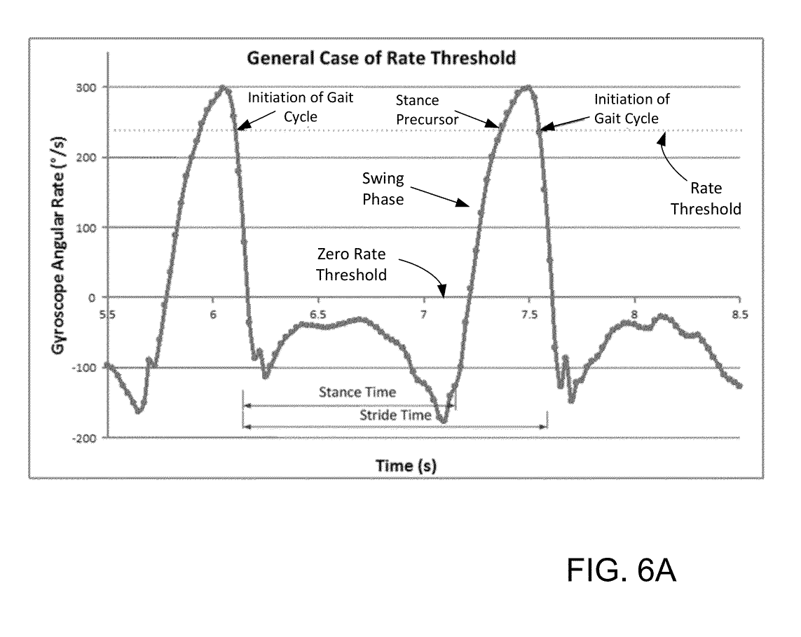

FIG. 6A shows a more general solution, wherein a rate threshold may be set anywhere along the steep portion of the swing phase. Depending on how far away from the zero rate threshold the rate threshold is set, a small error may occur in the stride time, and a larger error may occur in the stance interval, and in the resultant calculation of stride length using one of the several published stride length determination methods and equations. Conceptually, the threshold may be set anywhere along the steep portion of the downward slope of the swing phase. In the example of FIG. 6A, the threshold has been set at a rate of 240 degrees/second. Further, the steepness of the downward slope of the swing phase may be quantified by differentiating the rate data to find the angular acceleration, as shown in FIG. 6B. As a guideline, the threshold is generally set at a level such that the (absolute value of the) steepness of the slope (the angular acceleration) is greater than about 1000 degrees/sec². In the example angular acceleration data of FIG. 6B, the threshold is set to 2,800 degrees/sec². The data of FIG. 6B may be used to guide the selection of a threshold level, as in FIG. 6A. Alternately, the threshold can be applied directly to the acceleration data such as in FIG. 6B.

The disclosed methods and apparatus permit accurate and reliable determinations of instant in time of the start of each gait cycle for a much wider range of walking disabilities than the prior art, and thus provides an accurate measure of stride time, and many other gait parameters related to or synchronized by the start of the gait cycle. No matter how serious the disability, if the person to be tested can move one foot ahead of the other, and even where the foot never leaves the ground, the gyro measures with high reliability and accuracy just when the leg stops moving forward and starts moving backward, and when the leg starts moving forward after a pause. Unlike conventional approaches, transients caused by the physical interaction of the foot with the ground are not used or needed, and the forward or backward movement of the shank (ankle) can be used instead. Start of each gait can be detected for the entire range of walking disabilities, and relatively low (40 Hz) data sampling rates are adequate.

Systems can also use optical body motion capture techniques to measure stride time, and many other gait parameters related to or synchronized by the start of the gait cycle. Separate pressure plates are unnecessary, and data from an optical body motion capture system can be suitably processed for gait analysis.

In some examples, angular rate reversals available from the gyro of a first IMU attached to the shank or ankle are used to obtain suitable gait data. There are two angular rate reversals available from a gyro during walking. In some examples, one of the angular rate reversals is identified with the “initiation of forward swing” as the walker commences the forward movement of the shank, and the opposite angular rate reversal is identified as the “termination of forward swing” and defined as the start of the gait cycle as the walker's shank begins a short backward swing a few millisecond before physical heel strike. The rate of change of a gyro output is at a maximum at these points, and so the time of rate reversal can be determined with great accuracy by linear interpolation using gyro output sample points with both positive and negative angular rate values centered roughly around a zero rate. In other examples, times can be estimated as those associated with rates that are 1%, 5%, 10%, or 20% of a maximum rate.

In other embodiments, angular rate reversals of a first IMU are detected and used to synchronize the processing of data from one or more subsidiary IMUs. Stride length can be estimated using Aminian's pendulum model with Aminian's toe-off event and the termination of forward swing (angular rate reversal) event as disclosed herein. Stride length computation typically is based on detection of both of these gait events for at least one gait cycle. Angular rate reversals of a first IMU can be used to directly measure or to synchronize the measurement of any or all of the following gait characteristic: Stridemarkers such as start of gait cycle (forward swing termination), toe lift, forward swing initiation, and timing data such as stride time, step time (seconds), stance time (seconds or % of stride time). swing time (seconds), forward swing time (seconds), miscellaneous parameters such as stride length (meters), stride speed (meters/second), gyro peak rate (degrees/second), slope at heel strike (degrees/sec/sec), gyro drift estimate (degrees/second), and angular data such as knee joint range-of-motion (degrees), shank range-of-motion (degrees), thigh range-of-motion (degrees),

In other examples, angular rate reversals of a first IMU are used to allow the measurement of synchronized lateral movement data from additional IMUs. In some examples, angular rate reversals of a first IMU are used to allow the measurement of balance and fall-prediction parameters from additional IMUs. As noted above, the disclose systems and methods do not require IMUs, but can be based on any source of angular rate data such as an optical motion capture system.

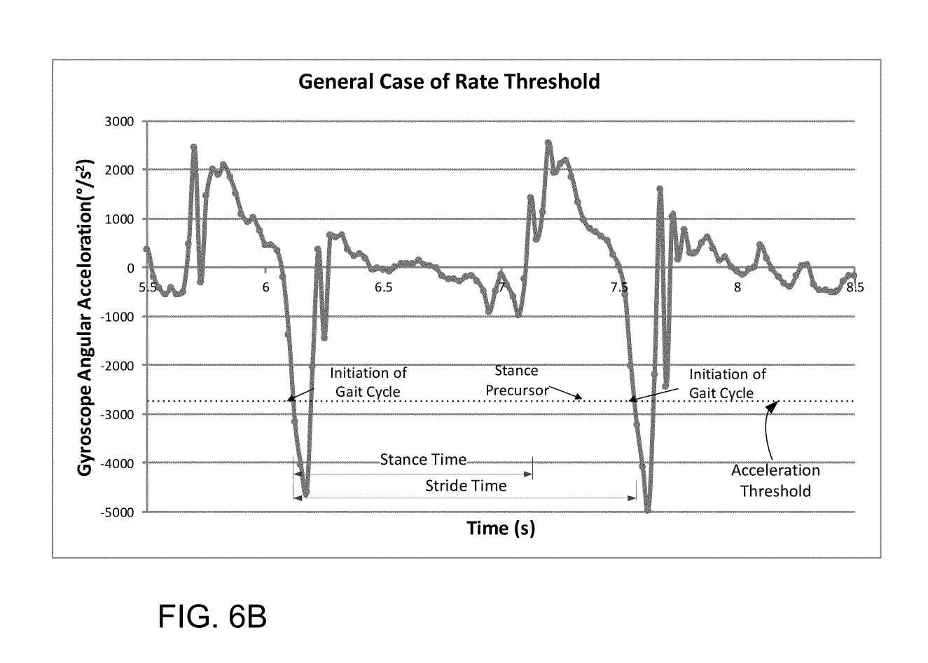

A representative body-mounted sensor system is illustrated in FIG. 7. Sensors are secured to a subject's shins and thighs, and rotational and other data is communicated via wireless link to a base station. A computer such as a lap top computer is coupled to the base station so as to receive and process the received angular data and estimate angular speeds and accelerations as needed, determine statistics of gait parameters, and to provide a user interface for user selection of parameters such as threshold levels.

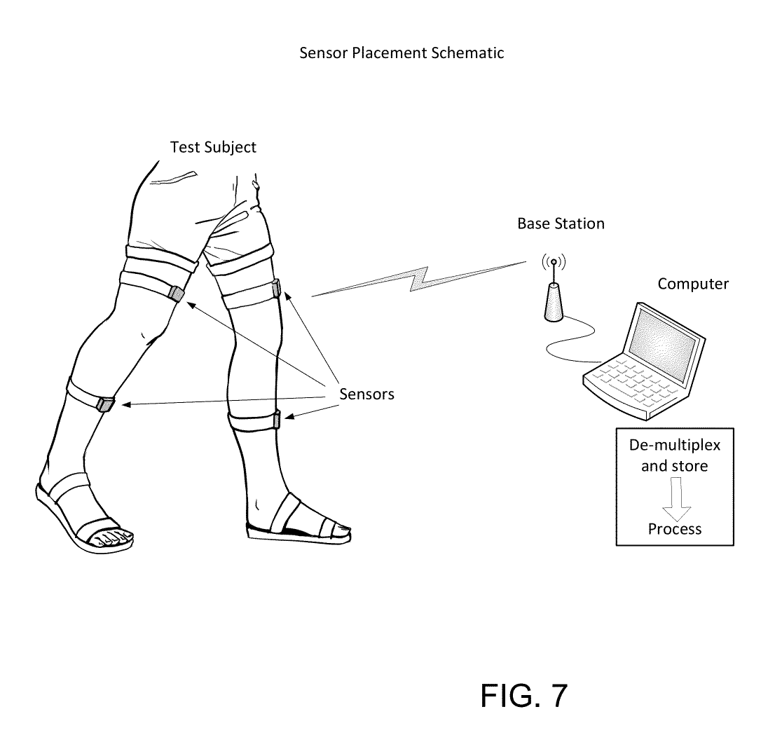

Gait analysis performed by the system of FIG. 7 can include some or all of the following as illustrated in FIGS. 8A-8C. As shown in FIG. 8A, gyro data (angular speed data or “G”) is input and differentiated at 802, and zero crossings in the derivative are identified at 804. A second derivative is obtained as 806 and magnitudes of the second derivative established at 808. Based on the derivatives, magnitude peaks are classified as positive or negative to determine troughs and peaks at 810. In addition, zero crossings in the gyro data are determined at 812.

Referring to FIG. 8B, an amplitude threshold value and previously identified peaks are input to an amplitude filter 822 that removes positive peaks below the amplitude threshold value. The remaining peaks are averaged at 824 and peaks having amplitudes less than ½ the mean value are removed at 826, and swing peaks are output.

Referring to FIG. 8C, at 842 first zero angular speed (G) crossings before each swing peak are found to determine a time associated with Initiation of Forward Swing. First zero angular speed crossings after each peak are determined at 844 as Termination of Forward Swing times. After the Initiation of Forward Swing times are found, prior zero crossings of dG/dt are found as Toe Lifts at 846. Heel strikes are determined at 848 as first zero crossings in G after troughs.

After processing a gait record as shown in FIGS. 8A-8C, stance times, stride times, forward swing times, heel strike times, and toe lift times can be estimated and means, standard deviations and other statistical parameters estimated. Processing is based on a peaks defined by dG/dt=0 and a peak threshold which can be varied.

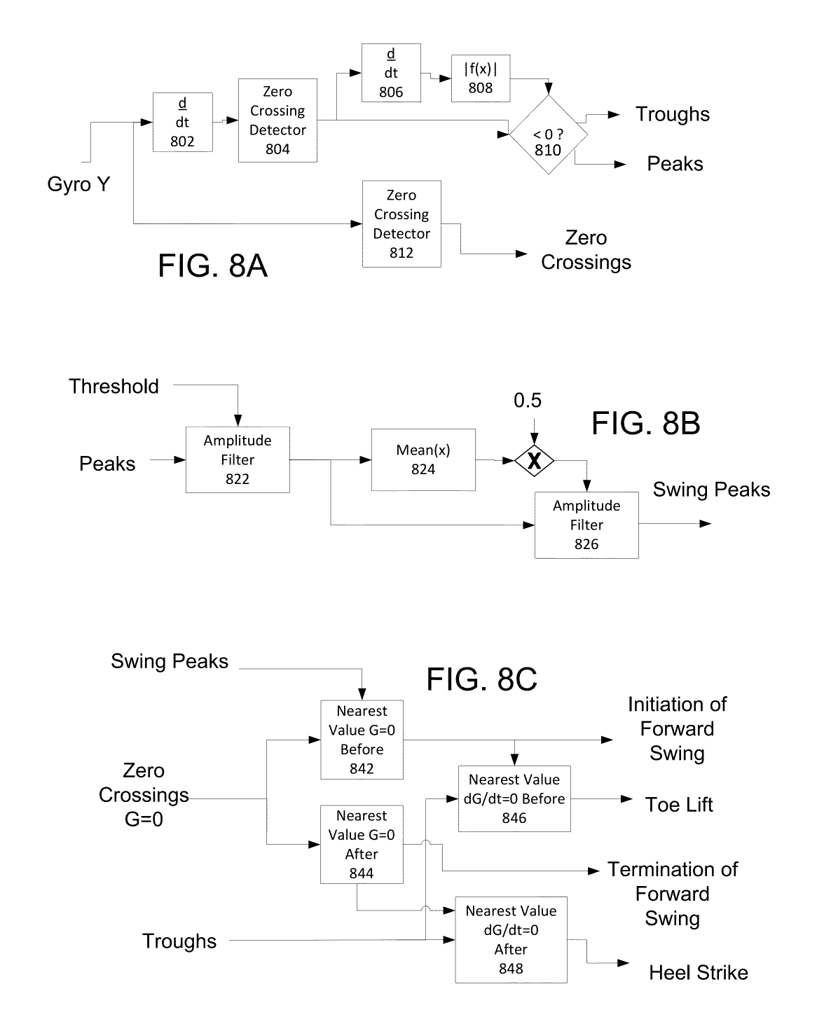

FIG. 9 illustrates a representative system 900 for the acquisition of gait data. Such a system can be referred to as an inertial measurement unit (IMU). The IMU 900 includes three Micro-Electro-Mechanical Systems (MEMS) sensors 902, 904, 906, which comprise two gyroscope ICs, an accelerometer IC, and a magnetometer IC, respectively. A receiver/transmitter 910 can be implemented as, for example, 915 MHz Industrial, Service and Medical (ISM) band two-way radio transceiver. This band generally exhibits a lower path loss factor than the 2.4 GHz ISM band. The receiver/transmitter 910 can be provided by a Nordic Semiconductor nRF905 transceiver that features proprietary packet handling capabilities which significantly reduce processing load for an onboard microcontroller 912. The peak gain of a radio link omni-directional antenna used in communications is generally arranged to be in a plane perpendicular to the X axis.

A 40 Hz data sampling rate can be used. A single-chip Rx/Tx can provide for longer battery life, and the magnetometer 906 is generally not sampled while transmitting. The 40 Hz sampling rate is also consistent with the human movement spectral range. Two sets of data are assembled into packets, and are transmitted at a rate of 20 Hz to a base station via a 915. The base station demodulates the data stream and transmits the re-constituted data to a computer via a USB port or other data connection. Data are conveniently expressed in a count format, ranging from 0 to 4095. For wireless systems, packet loss processing should be implemented to compensate occasional packet loss associated with temporary loss of communication links. Packets can be sequentially numbered so that gaps in a sequence can be detected, and each packet is time-stamped upon reception.

The disclosed methods and apparatus can be used in assessing and providing therapy for patients with cerebral palsy (CP), Parkinsonism, muscular dystrophy, osteoarthritis, rheumatoid arthritis, lower limb amputations, head injury, myelodysplasia, multiple sclerosis, spinal cord injury, or to assess aging patients. In one example, gait data was collected based on a series of measurements on severely disabled persons, including 6 persons with advanced Parkinson's Disease (PD), ages 74 to 84, and one 58 year old person with Cerebral Palsy (CP). The start of each gait cycle event was correctly detected for all test subjects, including a person with PD with a shuffling gait who could not walk without a walker, and whose feet never left the ground. The test subject with CP has a highly irregular gait but the start of gait was correctly identified. In addition, the probability of the false detection of gate initiation was determined to be 0%. A summary of the associated data is provided below in Table 1.

TABLE 1
Gait start detection for all subjects.

| | All Subjects | PD Total | PD Fallers | CP |
|---|---|---|---|---|
| Total Gait Cycle Start Events | 936 | 399 | 161 | 53 |
| Correct Detection | 100% | 100% | 100% | 100% |
| False Detection | 0% | 0% | 0% | 0% |

Gait analysis can also be used for fall risk assessment, typically based on estimation of the variability of the stride time and defined as the stride time standard deviation (SD). See, for example, Hausdorff et al., “Gait variability and fall risk in community-living older adults: a 1-year prospective study,” Archives of Physical Medicine and Rehabilitation 82:1050-6 (2001), which is incorporated herein by reference. Stride length is the second most important indicator of fall risk for persons with PD. See for, example, J. M. Hausdorff, “Gait dynamics in Parkinson's disease: common and distinct behavior among stride length, gait variability, and fractal-like scaling,” Chaos 19:026113 (2009), which is incorporated herein by reference.

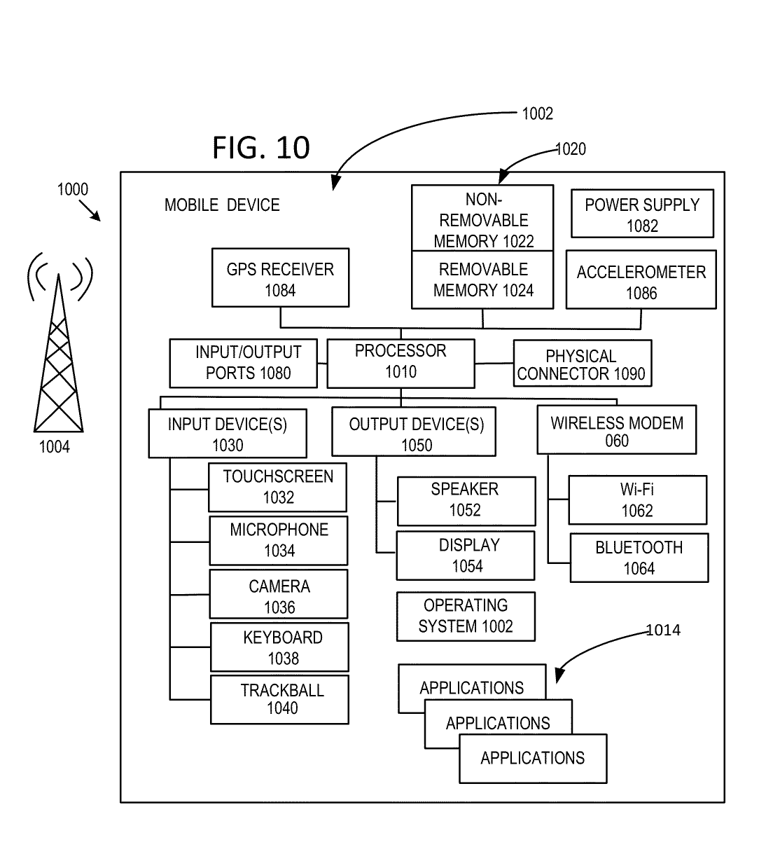

FIG. 11 shows graphical results obtained from a gait analysis system as disclosed herein for three persons with PD, all of whom participated in exercise classes for persons with PD. FIG. 11 shows measurements of the two most important fall-risk parameters, stride time SD and stride length, and includes the mean stride time SD for persons with Parkinson's for both fallers and non-fallers, as defined by Hausdorff.

Subject A cannot move at all without falling down unless he uses his walker. Subject A walks with a shuffling gait, and his feet never leave the ground. With his walker, he tests as midway between the faller and the non-faller mean values, and reports only an occasional fall. He was tested at an exercise class for persons with PD. Although he has a shuffling gait, the disclosed methods and apparatus were successful in detecting gait-cycle-starts with all starts correctly detected and no false detections.

Subjects B and C were tested at the start and at the end of an 8 week PD exercise program, with the specific object of measuring improvement. Initially, both were well into the fall-risk region, as defined by Hausdorff (2009), even though both used walking aids. Subject B insisted on using a walker at the start of the exercise program for fear of falling, and was tested using a walker at the start and at the end of the exercise program. By the end of the 8 week program he felt so confident that he insisted on repeating the program-end test using just a cane. Although his stride length was slightly less than with a walker, his stride-time SD was further reduced.

Similarly, subject C had to use a cane at the start of the program, and was so tested at the start and at the end. But he felt so confident in his walking ability that he insisted on a re-test with no cane at all, again coming out even better than with the cane. Test results for subjects B and C based on the disclosed methods and apparatus quantitatively supported the personal judgments of both subjects, which was that their gait was steadier, and they were able to reduce their dependence on their original walking aids. In addition, based on the test results, their risks of falling appear reduced at the end of the exercise program.

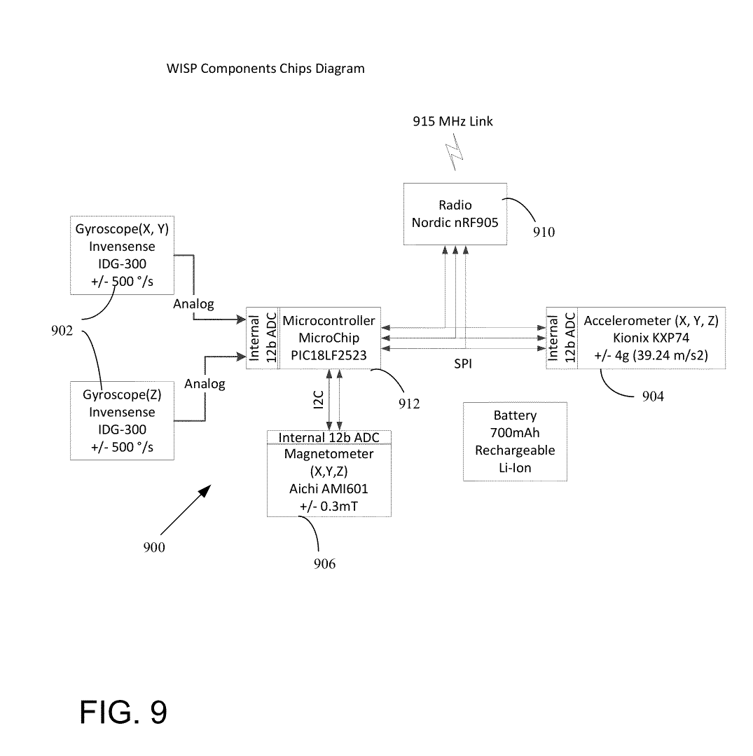

The representative methods and apparatus described above can be implemented using a variety of computing devices, methods, and hardware. FIG. 10 is a system diagram depicting an example mobile computing device 1000 that can be used to perform any of the methods described herein. The mobile computing device 1000 can include a variety of optional hardware and software components 1005. Generally, components 1005 can communicate with other components, although not all connections are shown, for ease of illustration. The computing device 1000 can be any of a variety of computing devices including mobile (e.g., cell phone, smartphone, handheld computer, laptop computer, notebook computer, tablet device, slate device, media player, Personal Digital Assistant (PDA), camera, video camera, etc.) and non-mobile (e.g., desktop computers, servers, gaming consoles, smart televisions) computing devices and can allow wired or wireless communication with one or more networks 1007, such as a Wi-Fi, cellular or satellite network.

The computing device 1000 can include a controller or processor 1010 (e.g., signal processor, graphics processing unit (GPU), microprocessor, ASIC, or other control and processing logic circuitry or software) for performing such tasks as signal coding, graphics processing, data processing, input/output processing, power control, and/or other functions. An operating system 1012 can control the allocation and usage of the components 1005 and support for one or more application programs 1014. The application programs 1014 can include common mobile computing applications (e.g., email applications, calendars, contact managers, web browsers, messaging applications) as well as other computing applications.

The mobile computing device 1000 can include memory 1020. Memory 1020 can include non-removable memory 1022 and removable memory 1024. The non-removable, or embedded memory 1022 can include RAM, ROM, flash memory, a hard drive, or other well-known memory storage technologies. The removable memory 1024 can include flash memory cards (e.g., SD (Secure Digital) cards), memory sticks, Subscriber Identity Module (SIM) cards, which are well known in GSM (Global System for Mobile Communication) systems, or other well-known memory storage technologies, such as “smart cards.” The memory 1020 can be used for storing data and/or computer-executable instructions for running the operating system 1012 and the application programs 1014 on the device 1000. Example data can include web pages, text, images, sound files, video data or other data sets to be sent to and/or received from one or more network servers or other devices by the mobile computing device 1000 via one or more wired or wireless networks. The computing device 1000 can also have access to external memory (not shown) such as external hard drives.

The computing device 1000 can support one or more input devices 1030, such as a touch screen 1032, microphone(s) 1034, camera(s) 1036, physical keyboard 1038 and/or trackball 1039 and one or more output devices 1040, such as a speaker(s) 1042, a display 1044 and 3D glasses 1046. Other possible output devices (not shown) can include piezoelectric or other haptic output devices. Any of the input devices 1030 and output devices 1040 can be internal to, external to, or removably attachable with the computing device 1000. External input and output devices 1030 and 1040 can communicate with the computing device 1000 via a wired or wireless connection. Some devices can serve more than one input/output function. For example, touchscreen 1032 and display 1044 can be combined in a single input/output device.

A wireless modem 1060 can be coupled to a wireless modem antenna 1062 and can support two-way communications between the mobile computing device 1000 and external devices, as is well understood in the art. The modem 1060 and the antenna 1062 are shown generically and can be a wireless cellular modem for communicating with a mobile cellular communication network. The wireless modem 1060 can comprise other radio-based modems such as a Wi-Fi modem 1063 or a Bluetooth modem 1064, each of which can be coupled to its own antenna (e.g., Wi-Fi antenna 1068, Bluetooth antenna 1069). The wireless modem 1060 is typically configured for communication with one or more cellular networks, such as a GSM network for data and voice communications within a single cellular network, between cellular networks, or between the mobile computing device and a public switched telephone network (PSTN).

The mobile computing device 1000 can further include at least one input/output port 1070 (which can be, for example, a USB port, IEEE 1394 (FireWire) port, and/or RS-232 port) comprising physical connectors 1072, a power supply 1074, a satellite navigation system receiver such as a GPS receiver 1075, a gyroscope 1076, an accelerometer 1077 and a compass 1078. The GPS receiver 1075 can be coupled to a GPS antenna 1079. The mobile computing device 1000 can additionally include an AM/FM antenna 180 coupled to an AM/FM receiver 185 for receiving radio signals broadcast by an AM/FM radio signal transmitter. The mobile computing device 100 can further include one or more additional antennas 190 coupled to one or more additional receivers, transmitters and/or transceivers 195 to enable various additional functions. For example, mobile computing device 1000 can include an additional antenna 1090 coupled to an additional receiver 1095 configured to receive and process a digital audio radio service (DARS) signal for output at the mobile computing device 1000 or an attached accessory.

Any of the disclosed methods can be implemented as computer-executable instructions or a computer program product. The computer-executable instructions or computer program products as well as any data created and used during implementation of the disclosed embodiments can be stored on one or more computer-readable media (e.g., non-transitory computer-readable media, such as one or more optical media discs, volatile memory components (such as DRAM or SRAM), or nonvolatile memory components (such as flash memory or hard drives)) and executed on a computer (e.g., any commercially available computer, including smart phones or other computing devices that include computing hardware). Computer-readable media does not include propagated signals. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it is to be understood that the disclosed technology is not limited to any specific computer language or program. For instance, the disclosed technology can be implemented by software written in C++, Java, Perl, JavaScript, Adobe Flash, or any other suitable programming language. Likewise, the disclosed technology is not limited to any particular computer or type of hardware. Certain details of suitable computers and hardware are well known and need not be set forth in detail in this disclosure.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, cable (including fiber optic cable), magnetic communications, electromagnetic communications (including RF, microwave, and infrared communications), electronic communications, or other such communication means.

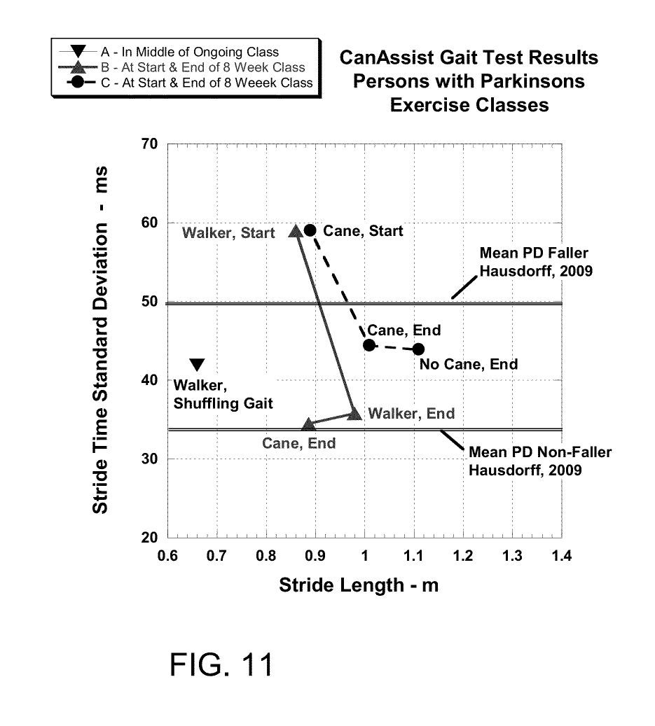

While the above examples focus on gait analysis for disabled subjects, in other examples such gait analysis can be used to assess athletes, effects of athletic shoes, fashion footwear such as high heels, orthotics, arch supports, hiking boots, court shoes on running, jogging, or walking. Barefoot subjects can be evaluated, and the effects of smooth, rough, slippery of other floor surfaces can be quantified using various shoe sole materials such a leather, suede, or rubber. FIG. 12 shows such data for one subject, where the stride time variation or uncertainty increases from barefoot to high heels, and for another subject where the uncertainty increases from running shoes, to mid-heels shoes and then to high heels.

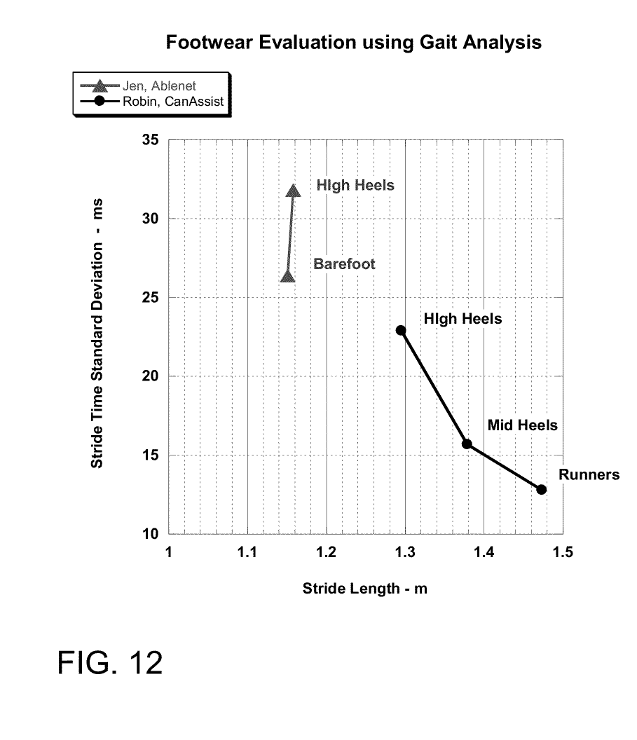

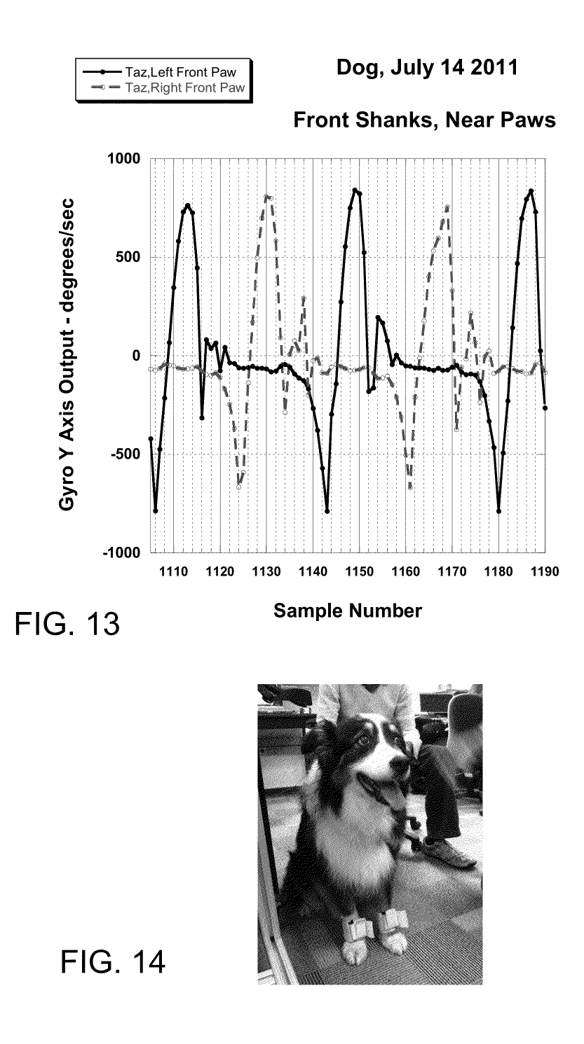

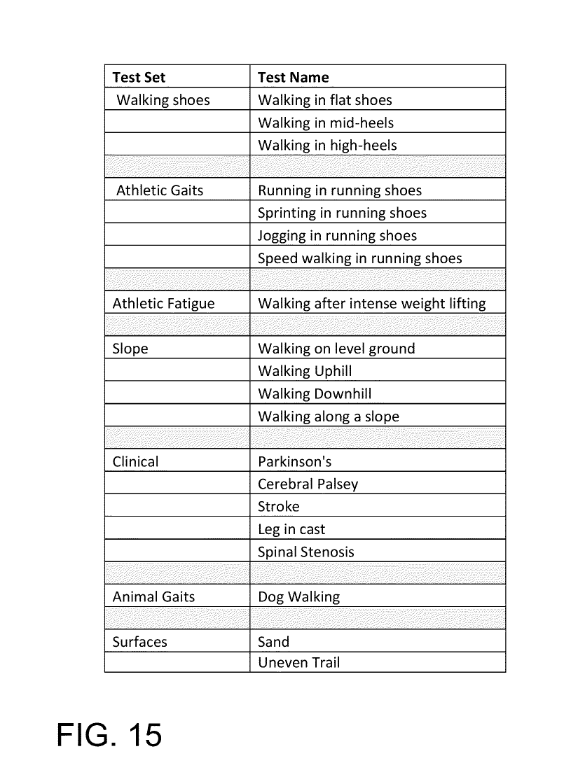

Fresh or fatigued subjects can be evaluated, and motion of the arms as well as the legs can be investigated. The disclosed methods and apparatus can also be applied to non-human subjects such as dogs and horses. FIG. 13 shows the gyroscope output waveforms for the left and right front paw of a walking dog. It will be noticed these bear a remarkable resemblance to those of a walking human shown in FIG. 2, and the same methods can be applied to a non-human subject such as a dog. FIG. 14 shows the location of the sensor units on the dog's front paws. FIG. 15 is a table of representative test subjects and conditions for which the disclosed methods and apparatus have been demonstrated to perform successfully.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of gait analysis, comprising:
obtaining an angular rate of a subject limb as a function of time during a gait cycle of the subject;
identifying at least one threshold crossing of the obtained angular rate during the gait cycle; and
estimating at least one gait characteristic based on the threshold crossing.

2. The method of claim 1, further comprising identifying at least two threshold crossings during the gait cycle, and estimating the at least one gait characteristic based on the threshold crossings.

3. The method of claim 2, wherein the gait characteristics are gait cycle time duration features.

4. The method of claim 1, wherein the at least one threshold crossing is based on an angular rate threshold computed from a local maximum angular acceleration.

5. The method of claim 1, wherein the at least one threshold crossing is based on a change in the angular rate from positive to negative or negative to positive.

6. The method of claim 1, wherein the at least one threshold crossing is associated with an angular rate reversal.

7. The method of claim 1, wherein the gait characteristic is associated with initiation of a gait cycle.

8. The method of claim 3, further comprising identifying an initiation of a second gait cycle based on the at least one threshold crossing.

9. The method of claim 1, further comprising assessing the subject based on the at least one gait characteristic.

10. The method of claim 1, wherein the threshold is an angular rate that is at least 80% of a maximum angular rate.

11. The method of claim 1, wherein the threshold is an angular rate that is at least 60% of a maximum angular rate.

12. The method of claim 1, wherein the threshold is an angular rate that is between 80% of a maximum angular rate and a zero angular rate.

13. The method of claim 1, wherein the threshold is determined by local maxima in angular acceleration, or differentiated angular velocity data.

14. The method of claim 1, wherein the threshold is associated with an angular acceleration of at least 50% of a maximum angular acceleration associated with a forward swing.

15. The method of claim 14, wherein the gait characteristic is associated with initiation of a gait cycle.

16. The method of claim 1, wherein the gait of the subject is associated with a disability, a disease, or a walking aid.

17. The method of claim 16, further comprising assessing the subject based on the at least one gait characteristic.

18. The method of claim 1, wherein the gait is a shuffling gate.

19. An apparatus, comprising:
at least one sensor configured to provide an angular rate associated with a shank at a plurality of times during at least one gait cycle; and
a processor configured to receive the angular rate at the plurality of times and based on a rate threshold, estimate a gait characteristic.

20. The apparatus of claim 19, wherein the gait characteristic is a gait cycle initiation time.

21. The apparatus of claim 19, wherein the at least one sensor is a gyroscopic sensor configured to be secured to the shank.

22. The apparatus of claim 19, wherein the sensor is an image capture sensor configured to produce the angular rate at the plurality of times based on a series of images of at least the shank.

23. The apparatus of claim 19, wherein the at least one gait characteristic is a stride length, stride duration, or a limb range of motion.

24. The apparatus of claim 19, wherein the at least one gait characteristic is a variation in at least one gait property.

25. The apparatus of claim 24, wherein the variation is associated with a variance of the at least one gait property.

26. At least one computer-readable medium comprising computer-executable instructions for the method of claim 19.

27. The method of claim 19, further comprising determining a stride time and stride length for a shuffling gait based on the gait characteristic.

28. A method of assessing cyclical motion, comprising:
obtaining an angular rate associated with the motion for at least one cycle;
identifying at least two angular rate reversals about a threshold value; and
characterizing the motion based on the identified angular rate reversals about the threshold value.

29. The method of claim 28, wherein the cyclical motion is a gait, and the angular rate is associated with motion of a shank.

30. The method of claim 28, wherein the cyclical motion is associated with the movement of an animal or of a mechanical device, or with swimming or cycling.

31. The method of claim 28, wherein the threshold is associated with an angular acceleration that is at least 25% of a maximum angular acceleration.

32. The method of claim 28, wherein the threshold is 25%, 50%, or 75% of a maximum obtained angular rate.

33. The method of claim 28, wherein the cyclical motion is a gait, and further comprising providing an assessment of a subject, a walking surface, or footwear.
